(19) United States
(12) Patent Application Publication
(10) Pub. No.: US 2026/0094668 A1
(43) Pub. Date: Apr. 2, 2026

(54) METHOD OF ANALYZING TUMOR SITE-SPECIFIC GENE MUTATION AND AN APPARATUS THEREFOR

(71) Applicant: CANON MEDICAL SYSTEMS CORPORATION, Otawara-shi (JP)

(72) Inventors: Asateru KIMURA, Yokohama (JP); Kosuke ARITA, Kawasaki (JP)

(73) Assignee: CANON MEDICAL SYSTEMS CORPORATION, Otawara-shi (JP)

(21) Appl. No.: 19/342,984

(22) Filed: Sep. 29, 2025

(30) Foreign Application Priority Data
Oct. 2, 2024 (JP) 2024-173877

(51) Int. Cl. G16B 20/20; A61K 45/06; G16B 30/00; G16H 10/60; G16H 20/10; G16H 30/20

(52) U.S. Cl. CPC G16B 20/20; A61K 45/06; G16B 30/00; G16H 10/60; G16H 20/10; G16H 30/20

(57) ABSTRACT

A method of analyzing gene mutation according to an embodiment, includes acquiring first gene mutation information identified by a ctDNA test in a subject, second gene mutation information output by analyzing an image of a first imaging target site in the subject, and third gene mutation information output by analyzing an image of a second imaging target site different from the first imaging target site in the subject; and determining whether a tumor-specific gene mutation included in the first gene mutation information is associated with a gene mutation in the first imaging target site included in the second gene mutation information, or a gene mutation in the second imaging target site included in the third gene mutation information.

### CROSS REFERENCE TO RELATED APPLICATIONS

This application is based upon and claims the benefit of priority from the prior Japanese Patent Applications No. 2024-173877, filed on Oct. 2, 2024, the entire contents of which are incorporated herein by reference.

### FIELD

The embodiments disclosed in this description and the drawings relate to a method of analyzing tumor site-specific gene mutation and an apparatus therefor.

### BACKGROUND

As tumors grow and evade the immune system, gene mutations are commonly accumulated. Certain cancers, such as lung cancer, bladder cancer, breast cancer, and melanoma, may contain 500 or more mutations. There are specific genomic loci in various cancers, known to be frequently mutated, commonly referred to as “hotspots”, such as a KRAS gene mutation commonly observed in colon and lung cancer and there are other “long tail” hotspot mutations in various cancer genes. Other genes with a concentration of mutational hotspots include BRAF, found in 50% of patients with melanoma (90% of these mutations are V600E); the BCR/Abl translocation, found in 95% of patients with CML; IDH1, found in 70%-90% of patients with glioma/glioblastoma; and a p53 mutation, found in many cancers. Detecting these gene mutations will enable targeted drug administration, treatment monitoring, early cancer screening, etc. to be carried out with ease and high accuracy.

As a method of detecting such gene mutation, liquid biopsy, a diagnostic method using patients' body fluids, has attracted attention and can be applied to the selection of diverse treatment methods and even early detection. Liquid biopsy is an alternative to surgery and biopsy specimen collection from primary and metastatic foci, which require invasive procedures, and enables repeated collections of samples in a less invasive manner to bring advantages of real time grasping of changes in tumor characteristics caused by treatment and also covering “tumor heterogeneity.” In liquid biopsy, though various components can be the measurement target, from the viewpoint of enabling highly accurate detection, circulating tumor DNA (ctDNA) is often selected as the measurement target.

Circulating tumor DNA, ctDNA, refers to tumor DNA that is present in body fluids such as blood and cerebrospinal fluid and is released outside the cells. ctDNA is usually mixed with free DNA originated from normal cells in the blood, also known as cfDNA (cellfree DNA). ctDNA can be easily obtained from blood etc., and thus enables the minimally invasive acquisition of tumor-originated DNA and also the highly accurate prediction of gene mutation.

In contrast, when a patient has multiple tumors, necrotic cells originated from the multiple tumors are mixed in body fluids including blood. Even if a ctDNA test is performed using this body fluid as a measurement sample and a gene mutation in a tumor can be identified, it may be difficult to identify which tumor the gene mutation originates from. In general, although metastatic tumors in the early stages have the same gene mutation as that in the tumor at the primary site, they acquire their own unique gene mutations with worsening and progressing, and thus in the case of a metastasized tumor, it may also be difficult to identify which tumor a gene mutation originates from. Accordingly, when a patient has multiple tumors, ctDNA testing alone cannot accurately identify the gene mutation specific to each tumor, which may cause concerns of disadvantage to the patient: for example, the patient may not be supplied with a drug specifically effective against each tumor.

### BRIEF DESCRIPTION OF THE DRAWINGS

FIG. 1 shows a diagram demonstrating an example of the flow of a method of analyzing a gene mutation according to a first embodiment.

FIG. 2 shows the results obtained by carrying out the method of analyzing gene mutation according to the first embodiment in a patient with lung cancer and pancreatic cancer.

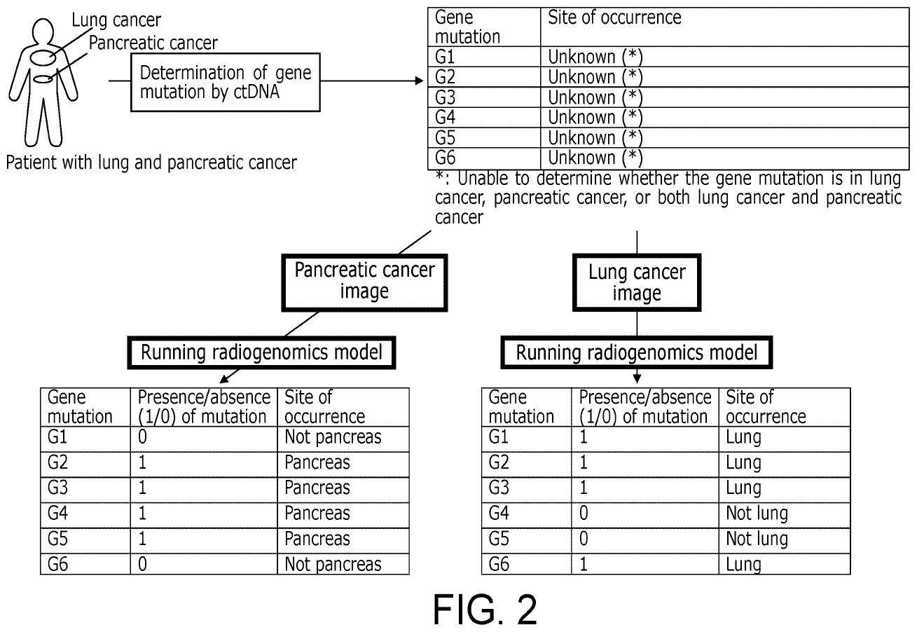

FIG. 3 shows the results obtained by carrying out the method of analyzing gene mutation according to the first embodiment in a patient with lung cancer, pancreatic cancer, and other undetected tumors.

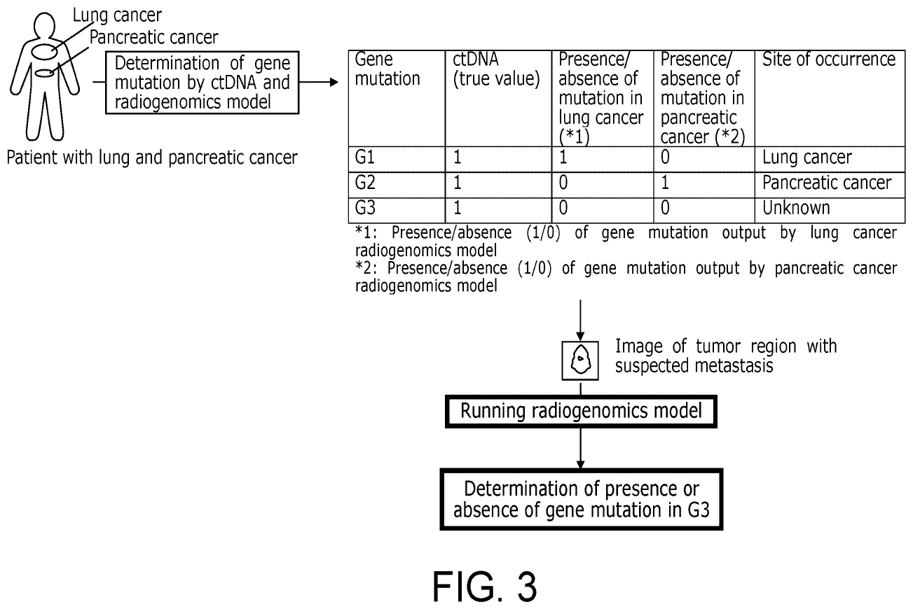

FIG. 4 shows a diagram demonstrating the results of evaluating the therapeutic effect of a drug on a patient with tumors by carrying out the method of analyzing gene mutation according to the first embodiment.

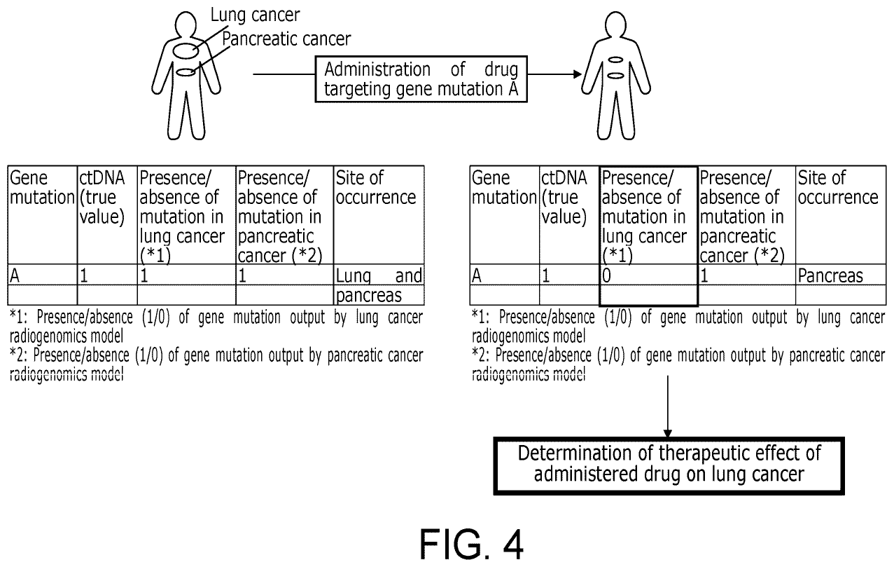

FIG. 5 is a block diagram illustrating an example of configuration of the analyzing system according to the third embodiment.

FIG. 6 is a block diagram illustrating an example of configuration of the analyzing apparatus according to the third embodiment.

### DETAILED DESCRIPTION

Hereinafter, embodiments of a method of analyzing gene mutation and an apparatus for analyzing gene mutation will be described in detail with reference to the drawings.

### First Embodiment

The method of analyzing gene mutation according to the first embodiment includes acquiring first gene mutation information identified by a ctDNA test in a subject, second gene mutation information output by analyzing an image of a first imaging target site in the subject, and third gene mutation information output by analyzing an image of a second imaging target site different from the first imaging target site in the subject, and determining whether a tumor-specific gene mutation included in the first gene mutation information is associated with a gene mutation in the first imaging target site included in the second gene mutation information, or a gene mutation in the second imaging target site included in the third gene mutation information. The method may be an in vitro method, a diagnostic aid, etc.

The method of analyzing gene mutation according to the first embodiment includes acquiring gene mutation information identified by a ctDNA test in a subject, gene mutation information output by analyzing an image of a first imaging target site in the subject, and gene mutation information output by analyzing an image of a second imaging target site different from the first imaging target site in the subject.

The “ctDNA test” in the method of analyzing gene mutation according to the first embodiment is not particularly limited as long as it is a test capable of detecting tumor-specific gene mutations (e.g., point mutations, indel mutations, HBV integration, etc.) in ctDNA, and can be performed in accordance with known methods, for example, by a PCR-based hotspot mutation detection method, capture sequencing, etc.

The gene mutation information identified by the “ctDNA test” in the method of analyzing gene mutation according to the first embodiment includes information on tumor-specific gene mutation in a subject targeted for the “ctDNA test.” The format of such information output is not particularly limited, and may be, for example, a record of determination for the presence or absence of tumor-specific gene mutation for each specific region (e.g., gene locus) of the subject's genome.

The method of obtaining the results of the “ctDNA test” in the method of analyzing gene mutation according to the first embodiment is not particularly limited, and the person implementing the method may perform the test himself/herself to obtain the test results, or, if an external institution is available for properly performing the “ctDNA test,” the test may be entrusted to such an institution to obtain the test results. Furthermore, if previous test results are available, those test results may be used as a reference.

The measurement sample used in the “ctDNA test” in the method of analyzing gene mutation according to the first embodiment is not particularly limited as long as it contains ctDNA, and is preferably one that can be obtained from a subject in a minimally invasive manner. Specific examples of such measurement samples include blood, urine, cerebrospinal fluid, saliva, semen, bile, pleural fluid, ascites, stool, vaginal fluid, etc., and blood is preferable.

The “subject” in the method of analyzing gene mutation according to the first embodiment is not particularly limited as long as it has multiple tumors, and examples thereof include mammals such as primates including humans and chimpanzees, pet animals such as dogs and cats, livestock animals such as cows, horses, sheep and goats, and rodents such as mice and rats. The “subject” in the method of analyzing a gene mutation according to the first embodiment is preferably a human.

The “image” in the method of analyzing a gene mutation according to the first embodiment is not particularly limited, and examples thereof include a radiological image, a magnetic resonance image, and an ultrasonic image, and a radiological image is preferable. The method of taking this image is not particularly limited, and for example, an operator such as a doctor (e.g., a radiologist) or a diagnostic radiologist may take the image by operating an apparatus capable of taking images.

In the method of analyzing gene mutation according to the first embodiment, the “image” may be divided into a plurality of regions for analysis: for example, the “image” may be divided into a grid (squares) of a predetermined shape and scale. The grid may be, for example, one-to-one with respect to a pixel, or may include multiple pixels within the grid.

When the “image” in the method of analyzing a gene mutation according to the first embodiment is a planar image, the grid is, for example, a square. The grid may be of a shape other than a square. The grid may be, for example, a shape enclosed by straight lines, such as a rectangle or a regular polygon other than a square. Furthermore, when the “image” in the method of analyzing gene mutation according to the first embodiment is a three-dimensional image, the grid is, for example, a cube, and may be a rectangular prism other than a cube or may have some other shape.

In the method of analyzing gene mutation according to the first embodiment, the method of analyzing the image of imaging target site is not particularly limited, and can be performed by, for example, calculating image feature amount. In this case, the image feature amount may be calculated for each image, or may be calculated for each of a plurality of regions. The image feature amount is not particularly limited as long as enabling determination whether or not the imaging target site has a gene mutation, and may be, for example, various image feature amounts used in radiogenomics (radiology genomics: a science that systematically deals with large amounts of information related to genes).

In the method of analyzing gene mutation according to the first embodiment, gene mutation information output by analyzing an image of an imaging target site includes information enabling determination of the presence or absence of gene mutation in the imaging target site. The format of this output is not particularly limited, and may be a description for each specific region of the subject's genome (e.g., a gene locus), such as a determination of the presence or absence of gene mutation, a classification of the gene mutation, an estimated probability of the gene mutation, or a combination of these.

The method of calculating the estimated probability of this gene mutation is not particularly limited, and the calculation can be performed in accordance with a known method: for example, the calculation can be performed by further subjecting the results output by an appropriate radiogenomics model to a probability calibration process.

In the method of analyzing gene mutation according to the first embodiment, an image analysis is performed for a first imaging target site and a second imaging target site different from the first imaging target site. Here, the case “different from” includes, as well as a case where the first imaging target organ and the second imaging target organ are not the same organ, a case where, though the first imaging target organ and the second imaging target organ are the same organ, the gene mutation information output by analyzing an image in the first imaging target site is not the same as that in the second imaging target site different from the first imaging target site.

The “imaging target site” in the method of analyzing gene mutation according to the first embodiment is not particularly limited as long as it is a site where a tumor is or was present in the past. Such tumors may be primary or metastatic. Examples of such tumors include carcinomas (malignant epithelial tumors) and sarcomas (malignant non-epithelial tumors), and specific examples include lung cancer, colon cancer, breast cancer, prostate cancer, liver cancer, brain cancer, kidney cancer, ovarian cancer, stomach cancer, skin cancer, bone cancer, pancreatic cancer, glioma, glioblastoma, hepatocellular carcinoma, papillary renal carcinoma, head and neck squamous cell carcinoma, and melanoma.

The method of analyzing gene mutation according to the first embodiment includes determining whether a tumor-specific gene mutation included in gene mutation information identified by a ctDNA test is associated with a gene mutation in a first imaging target site or a gene mutation in a second imaging target site. The determination may be performed as follows: for example, when a gene mutation is determined to be present by ctDNA testing and the same gene mutation is also determined to be present in the first imaging target site or the second imaging target site, the gene mutation will be determined to be present in the tumor in the imaging target site determined to have the same gene mutation, and the gene mutation will be determined to be not present in the tumor in the imaging target site determined not to have the same gene mutation.

The method of analyzing gene mutation according to the first embodiment may further include correcting information on the presence or absence of gene mutation in an imaging target site, which is included in at least one of the gene mutation information output by analyzing an image of a first imaging target site and the gene mutation information output by analyzing an image of a second imaging target site different from the first imaging target site.

The correction of information on the presence or absence of gene mutation in the imaging target site may include optimization of the gene mutation information that is output based on a predetermined threshold value used for determining the presence or absence of the gene mutation. Such optimization can be performed by correcting a determination result of, for example, presence of the gene mutation to absence when the estimated probability output of the gene mutation is smaller than a normal threshold value.

Furthermore, the correction of information on the presence or absence of gene mutation in the imaging target site may include optimization of the threshold value. Such optimization can be performed, for example, on a trained radiogenomics model using image data, different from those used for training, labeled with the presence/absence of gene mutation and then determining an optimal threshold value based on the values calculated. In addition, for example, for a gene mutation in which all of the determination results of the presence or absence of gene mutation output by each radiogenomics model are no mutation, there can be also used the estimated probability of the gene mutation output by another radiogenomics model as a threshold value.

Furthermore, when the analysis of the image of imaging target site includes the calculation of image feature amount, the correction of information on the presence or absence of gene mutation in the imaging target site may include optimization of the gene mutation information output based on a correction coefficient. Such optimization can be performed, for example, on a trained radiogenomics model using image data, different from those used for training, labeled with the presence/absence of gene mutation and then determining an optimal correction coefficient based on the values calculated.

The method of analyzing gene mutation according to the first embodiment includes determining whether a tumor-specific gene mutation included in gene mutation information identified by a ctDNA test is associated with a gene mutation in a first imaging target site or a gene mutation in a second imaging target site. When neither the gene mutation in the first imaging target site nor the gene mutation in the second imaging target site is determined to be associated with the tumor-specific gene mutation, the method of analyzing gene mutation according to the first embodiment may include showing the tumor-specific gene mutation to be present in a new site different from the first imaging target site and the second imaging target site.

The method of analyzing gene mutation according to the first embodiment enables identification of tumor site-specific gene mutation. Change in the identified gene mutation from presence to absence by administration of a drug treatment to the tumor allows prediction that the drug will have a therapeutic effect on the tumor. Accordingly, the method of analyzing gene mutation according to the first embodiment may further include determining the following: at a first determination time, the tumor-specific gene mutation is determined to be present in any imaging target site; at a second determination time of an arbitrary time after the first determination time, the tumor-specific gene mutation is determined to be not present in the imaging target site; and thus a drug applied to the subject until the second determination time is determined to have a therapeutic effect against a disease associated with the tumor-specific gene mutation.

Examples of such drugs include molecular targeted drugs and cancer immune checkpoint inhibitors.

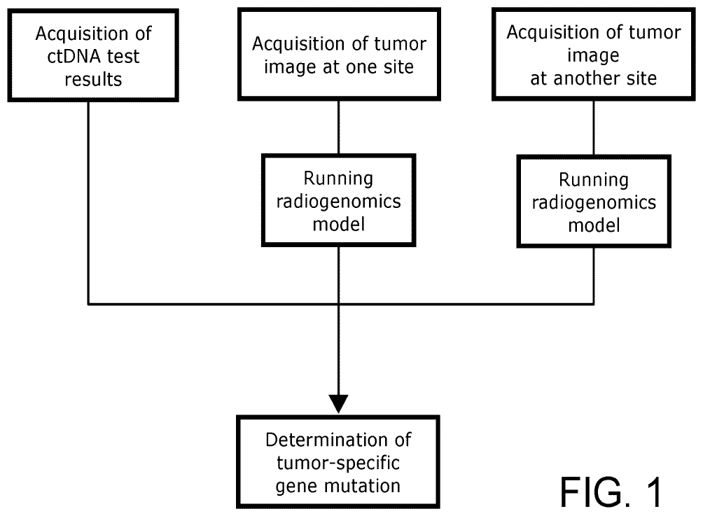

FIG. 1 shows a diagram demonstrating an example of the flow of a method of analyzing a gene mutation according to a first embodiment. The method of analyzing gene mutation shown in FIG. 1 includes the steps for a patient with multiple tumors: (1) obtaining ctDNA test results, (2) obtaining tumor images at one site and running a radiogenomics model, (3) obtaining tumor images at another site and running a radiogenomics model, and (4) determining tumor-specific gene mutation. The step (1) may subsequently include acquiring first gene mutation information including tumor-specific gene mutation. The step (2) may subsequently include acquiring second gene mutation information including a gene mutation at the one site. The step (3) may subsequently include acquiring third gene mutation information including a gene mutation at the another site. The determination in step (4) may include confirming whether a tumor-specific gene mutation included in the first gene mutation information is associated with a gene mutation in the one site included in the second gene mutation information, or a gene mutation in the another site included in the third gene mutation information.

### Second Embodiment

In the second embodiment, treatment of one or more tumors in the subject can be performed based on data obtained by the method of analyzing gene mutation according to the first embodiment.

The method of treating one or more tumors in the subject according to the second embodiment includes acquiring first gene mutation information identified by a ctDNA test in a subject, second gene mutation information output by analyzing an image of a first imaging target site in the subject, and third gene mutation information output by analyzing an image of a second imaging target site different from the first imaging target site in the subject, determining whether a tumor-specific gene mutation included in the first gene mutation information is associated with a gene mutation in the first imaging target site included in the second gene mutation information, or a gene mutation in the second imaging target site included in the third gene mutation information, and, if determining a tumor-specific gene mutation included in the first gene mutation information is associated with a gene mutation in the first imaging target site included in the second gene mutation information, or a gene mutation in the second imaging target site included in the third gene mutation information, then administering a therapeutically effective amount of drug to the subject determined to have one or more tumors whose presence is (are) confirmed by the gene mutation at the first imaging target site or at the second imaging target site based on the determination data.

In the method of treating one or more tumors in the subject according to the second embodiment, if determining a tumor-specific gene mutation included in the first gene mutation information is associated with a gene mutation in the first imaging target site included in the second gene mutation information, or a gene mutation in the second imaging target site included in the third gene mutation information, then, based on the determination data, therapeutically effective drugs are selected and used for treatment to the subject determined to have one or more tumors whose presence is (are) confirmed by the gene mutation at the first imaging target site or at the second imaging target site. The one or more tumors whose presence is (are) confirmed by the gene mutation at the first imaging target site or at the second imaging target site may be one or more tumors in the first imaging target site or in the second imaging target site.

Furthermore, in the method of treating one or more tumors in the subject according to the second embodiment, those skilled in the art can appropriately determine therapeutically effective drugs for the tumor according to the tumor type. In the method of treating one or more tumors in the subject according to the second embodiment, the drug can be selected according to the tumor type (for example, for lung cancer, cytotoxic anticancer drugs such as paclitaxel, molecular targeted drugs such as bevacizumab, or immune checkpoint inhibitors such as pembrolizumab; for pancreatic cancer, cytotoxic anticancer drugs such as gemcitabine, molecular targeted drugs such as olaparib, or immune checkpoint inhibitors such as pembrolizumab). The one or more drugs may be selected. The selected drug is administered to the subject in a therapeutically effective amount (an amount sufficient to achieve or at least partially achieve a desired therapeutic effect). The therapeutically effective amount can be determined according to a conventional treatment protocol.

In the method of treating one or more tumors in the subject according to the second embodiment, the method of administering the drug can be appropriately selected according to the drug type, and specific examples of such methods include, but are not limited to, oral administration, transdermal administration, intravenous administration, intramuscular administration, subcutaneous injection, nasal administration, inhalation administration, sublingual administration, and rectal administration.

The meanings and indications of the terms used in describing the second embodiment are the same as those used in describing the first embodiment. Therefore, for example, in the method of treating one or more tumors in the subject according to the second embodiment, the subject and the type of tumor are the same as those in the method of analyzing gene mutation according to the first embodiment.

### Third Embodiment

The method for analyzing a gene mutation according to the first embodiment described above may be executed in an apparatus for analyzing a gene mutation. Below will be the explanation on the differences from the first embodiment described above.

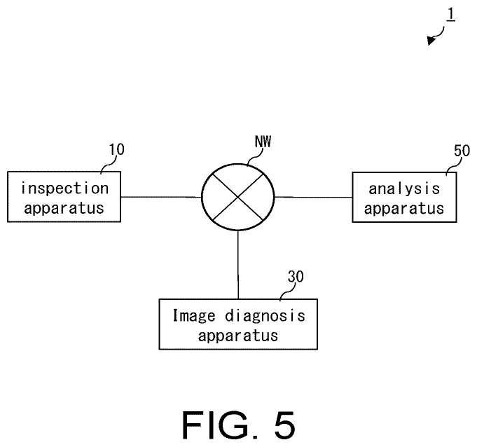

FIG. 5 is a block diagram illustrating an example of configuration of an analysis system according to a third embodiment. As shown in FIG. 5, the analysis system 1 is configured with an inspection apparatus 10, an image diagnosis apparatus 30, and an analysis apparatus 50. The inspection apparatus 10, the image diagnosis apparatus 30, and the analysis apparatus 50 are connected via a network NW to communicate with each other.

The inspection apparatus 10 is an apparatus to perform a ctDNA test. Also, the inspection apparatus 10 outputs a first gene mutation information identified by a ctDNA test. The inspection apparatus 10 may be configured to be able to transmit a first gene mutation information identified by a ctDNA test to the analysis apparatus 50 or a data storage apparatus (not shown in the drawings) which stores data of the first gene mutation information.

The image diagnosis apparatus 30 generates an image by imaging a subject. The image may be an image of a first imaging target site or an image of a second imaging target site. Then, image diagnosis apparatus 30 can transmit the generated image to the analysis apparatus 50 or an image storage apparatus (not shown in the drawings) which stores the image. The image diagnosis apparatus 30 may be an X-ray diagnosis apparatus, an X-ray imaging apparatus such as an X-ray CT apparatus, an ultrasonic diagnosis apparatus, or an MRI apparatus.

Also, the image diagnosis apparatus 30 according to the present embodiment may analyze an image of a first imaging target site in a subject and, then, generate a second gene mutation information output by analyzing the image of the first imaging target site in the subject. The image diagnosis apparatus 30 may be configured to be able to transmit the generated second gene mutation information to the analysis apparatus 50 or a data storage apparatus.

Furthermore, the image diagnosis apparatus 30 according to the present embodiment may analyze an image of a second imaging target site in a subject and, then, generate a third gene mutation information output by analyzing the image of the second imaging target site in the subject. The image diagnosis apparatus 30 may be configured to be able to transmit the generated third gene mutation information to the analysis apparatus 50 or a data storage apparatus.

The analysis apparatus 50 is an apparatus to analyze a gene mutation. The analysis apparatus 50 may be, for example, a cloud server on a network. The analysis apparatus 50 is not limited to a cloud server, and may be a server or a terminal device installed in a hospital.

The network NW is an information and communication network that uses telecommunications technology in general. The network NW may be a telecommunications network, a fiber optic communication network, a cable communication network, a satellite communication network, as well as an internet network or a wireless/wired local area network (LAN) such as a backbone network LAN of a hospital.

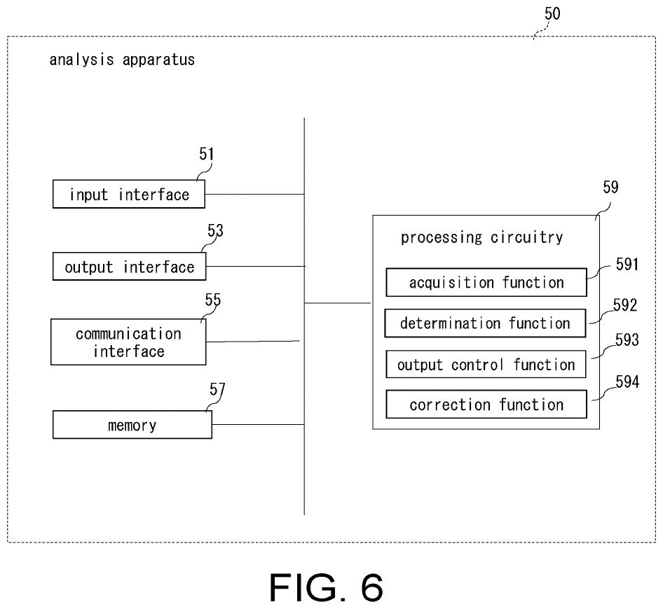

FIG. 6 is a block diagram illustrating an example of configuration of an analysis apparatus 50 according to a third embodiment. As shown in FIG. 6, the analysis apparatus 50 according to the third embodiment can be configured with an input interface 51, an output interface 53, a communication interface 55, a memory 57, and processing circurity 59. These multiple elements that configure the analysis apparatus 50 may be stored in a single housing or may be distributed into multiple housing as well.

The input interface 51 is a circuitry that receives various user input. The input interface 51 may be a mouse, a keyboard, a trackball, a manual switch, a foot switch, a button, a joystick, or the like.

The output interface 53 outputs various images or information. For instance, the output interface 53 may output a determination result as to whether the tumor-specific gene mutation included in the first gene mutation information is associated with a gene mutation in the first imaging target site included in the second gene mutation information, or a gene mutation in the second imaging target site included in the third gene mutation information, which is determined by the determination function 592 described later, a Graphical User Interface (GUI) to receive various user input, or the like. The output interface 53 may be configured by a liquid crystal display, a Cathode Ray Tube (CRT) display, a speaker, or the like. The output interface 53 is equivalent to an output in the present embodiment.

The communication interface 55 implements various telecommunications protocol according to the form of the network NW. The communication interface 55 realizes communication with a different apparatus via the network NW according to these various protocol. Specifically, in the present embodiment, the analysis apparatus 50 can be connected to the network NW via the communication interface 55 to communicate with the inspection apparatus 10 or the image diagnosis apparatus 30.

The memory 57 may be realized by a Random Access Memory (RAM), a semiconductor memory element such as a flash memory, a hard disk, an optical disk, or the like. In the present embodiment, the memory 57 may store the first gene mutation information, the second gene mutation information, the third gene mutation information, or the like.

The processing circuitry 59 is an arithmetic circuitry that performs various arithmetic operation. The processing circuitry 59 may be configured by a processor. The processing circuitry 59 according to the present embodiment may analyze the first gene mutation information, the second gene mutation information, or the third gene mutation information. Also, the processing circuitry 59 according to the present embodiment may analyze an image of a first imaging target site in the subject or an image of a second imaging target site in the subject.

Here, the word processor means a Central Processing Unit (CPU), a Graphics Processing Unit (GPU), an Application Specific Integrated Circuit (ASIC), a programmable logic device (a Simple Programmable Logic Device (SPLD), a Complex Programmable Logic Device (CPLD), or a Field Programmable Gate Array (FPGA)), or the like. The processor realizes functions by reading and executing a program stored in the memory 57. Note that, it is also possible to configure by directly incorporating the program inside the processor circuit instead of storing the program inside the memory 57. In this case, the processor realizes functions by reading and executing the program incorporated into the circuit. Note that the processor is not limited to be configured as a single circuit, but may be configured by combining multiple independent circuits to realize the function. It is also possible to combine the elements shown in FIG. 6 described later into one processor to realize the function.

As shown in FIG. 6, the processing circuitry 59 may have an acquisition function 591, a determination function 592, an output control function 593, and a correction function 594. The acquiring function 591 is equivalent to an acquisition according to the present embodiment; the determination function 592 is equivalent to a determination according to the present embodiment; the output control function 593 is equivalent to an output control according to the present embodiment; the correction function 594 is equivalent to a correction according to the present embodiment. The processing circuitry 59 according to the present embodiment may not have the correction function 594.

In the embodiment shown in FIG. 6, each processing function performed in the acquisition function 591, the determination function 592, the output control function 593, and the correction function 594, can be stored in the memory 57 in the form of a computer executable program. The processing circuitry 59 may be a processor that realizes functions related to each program by reading and executing the program from the memory 57. In other words, the processing circuitry 59 that has read each program now possess each function shown inside the processing circuitry 59 in FIG. 6. Note that, in FIG. 6, there is a single processing circuitry that realizes the acquisition function 591, the determination function 592, the output control function 593, and the correction function 594 inside a single processing circuitry 59, however, possible embodiments are not limited to this example. The processor may be configured by combining multiple independent processors to realize the function by letting each processor to execute each program.

The acquisition function 591 can acquire first gene mutation information identified by a ctDNA test in a subject, second gene mutation information output by analyzing an image of a first imaging target site in the subject, and third gene mutation information output by analyzing an image of a second imaging target site different from the first imaging target site in the subject. The acquisition function 591 according to the present embodiment may acquire first gene mutation information identified by a ctDNA test from the inspection apparatus 10 or the data storage apparatus via the network NW and the communication interface 55.

Also, the acquisition function 591 may acquire the image of the first imaging target site in the subject from the image diagnosis apparatus 30 or the data storage apparatus via the network NW and the communication interface 55. Then, the acquisition function 591 may analyze the image of the first imaging target site in the subject and acquire a second gene mutation information output by analyzing the image of the first imaging target site in the subject. Note that the acquisition function 591 may acquire a second gene mutation information output by analyzing the image of the first imaging target site in the subject from the image diagnosis apparatus 30 or another apparatus.

Furthermore, the acquisition function 591 may acquire the image of the second imaging target site in the subject from the image diagnosis apparatus 30 or the data storage apparatus via the network NW and the communication interface 55. Then, the acquisition function 591 may analyze the image of the second imaging target site in the subject and acquire a third gene mutation information output by analyzing the image of the second imaging target site in the subject. Note that the acquisition function 591 may acquire a third gene mutation information output by analyzing the image of the second imaging target site in the subject from the image diagnosis apparatus 30 or another apparatus.

The determination function 592 can determine whether a tumor-specific gene mutation included in the first gene mutation information is associated with a gene mutation in the first imaging target site included in the second gene mutation information, or a gene mutation in the second imaging target site included in the third gene mutation information.

Also, the determination function 592 may have functions to determine the following: at a first determination time, the tumor-specific gene mutation is determined to be present in any imaging target site; at a second determination time of an arbitrary time after the first determination time, the tumor-specific gene mutation is determined to be not present in the imaging target site; and thus a drug applied to the subject until the second determination time is determined to have a therapeutic effect against a disease associated with the tumor-specific gene mutation.

The output control function 593 can control the output interface 53 to output a determination result. The output control function 593 can control the output interface 53 to output a determination result as to whether the tumor-specific gene mutation included in the first gene mutation information is associated with a gene mutation in the first imaging target site included in the second gene mutation information, or a gene mutation in the second imaging target site included in the third gene mutation information.

Also, the output control function 593 may have functions to show the tumor-specific gene mutation to be present in a new site different from the first imaging target site and the second imaging target site when neither the gene mutation in the first imaging target site nor the gene mutation in the second imaging target site is determined to be associated with the tumor-specific gene mutation.

The correction function 594 can correct information on the presence or absence of gene mutation in an imaging target site included in at least one of the second gene mutation information and the third gene mutation information.

The apparatus for analyzing gene mutation according to the third embodiment includes an acquisition function to acquire first gene mutation information identified by a ctDNA test in a subject, second gene mutation information output by analyzing an image of a first imaging target site in the subject, and third gene mutation information output by analyzing an image of a second imaging target site different from the first imaging target site in the subject, and a determination function to determine whether a tumor-specific gene mutation included in the first gene mutation information is associated with a gene mutation in the first imaging target site included in the second gene mutation information, or a gene mutation in the second imaging target site included in the third gene mutation information.

The meanings and indications of the terms used in describing the third embodiment are the same as those used in describing the first embodiment.

Note that, in the analysis system 1 according to the third embodiment, the image diagnosis apparatus 30 is configured to generate a second gene mutation information output by analyzing the image of the first imaging target site in the subject and a third gene mutation information output by analyzing the image of the second imaging target site in the subject. However, an apparatus to generate a second gene mutation information and a third gene mutation information is not limited to the image diagnosis apparatus 30. In other words, an apparatus to generate a second gene mutation information and a third gene mutation information may be anything. The analysis apparatus 50 may analyze the image of the first imaging target site or the second imaging target site in the subject and generate the second gene mutation information or the third gene mutation information. Alternatively, another apparatus different from the analysis apparatus 50 may analyze the image of the first imaging target site or the second imaging target site in the subject and generate the second gene mutation information or the third gene mutation information.

The present invention will be specifically described based on the following examples, but the present invention is not limited to these examples.

Determining the Presence or Absence of Tumor-Specific Gene Mutation in Patient with Multiple Tumors

The ctDNA test results of patients with lung cancer and pancreatic cancer were obtained from a public database.

Images were obtained from organs where a tumor was predicted to be present based on the ctDNA test results, and the presence or absence of gene mutation in the tumor was determined using a radiogenomics model in the following procedure. Image feature amounts (Radiomics feature amount, etc.) were calculated from the training image data. These image feature amounts were used as input to train a radiogenomics model that classifies tumors as having or not having gene mutation. Thereafter, image feature amounts (Radiomics features, etc.) were calculated from the verification image data. These image feature amounts were then input into a trained radiogenomics system to classify the tumors as having or not having gene mutation.

The results of determining the presence or absence of gene mutation using the radiogenomics model were corrected using the following procedure. First, when the estimated probability of gene mutation output by each radiogenomics model (lung cancer, pancreatic cancer) was smaller than a normal threshold value, the output determination result was reversed. The threshold value used was a value determined for the trained radiogenomics model using image data, different from those used for training, labeled with the presence/absence of gene mutation. For gene mutation in which all of the determination results of the presence or absence of gene mutation output by each radiogenomics model are no mutation, the estimated probability of gene mutation output by another radiogenomics model was used as a threshold value, and when the estimated probability was greater than the normal threshold value, the output determination result was reversed.

In addition, the correction coefficient α for the estimated probability of gene mutation output by the radiogenomics model, was optimized using another image data labeled with the presence/absence of gene mutation, which are different from those used for training. The result of the determination of the presence or absence of gene mutation was derived from the magnitude relationship between the output estimated probability of gene mutation×correction coefficient and a threshold value. The results are shown in Table 1 below. In the gene mutation column in the table, “gene mutation present” is indicated as “1” and “gene mutation absent” is indicated as “0.”

TABLE 1
Results of determining presence or absence of gene
mutation using corrected radiogenomics model

| | | | | Presence/ | | | Presence/ |
|---|---|---|---|---|---|---|---|
| | | Presence/ | | absence of | Presence/ | | absence of |
| | | absence of | | gene | absence of | | gene |
| | | gene | | mutation | gene | | mutation |
| | | mutation | Threshold | output by | mutation | Threshold | output by |
| | | output by | value for | corrected | output by | value for | corrected |
| | | lung | lung | lung | pancreatic | pancreatic | pancreatic |
| | | cancer | cancer | cancer | cancer | cancer | cancer |
| | ctDNA | radio- | radio- | radio- | radio- | radio- | radio- |
| Gene | (true | genomics | genomics | genomics | genomics | genomics | genomics |
| mutation | value) | model (*1) | model | model (*2) | model (*1) | model | model (*2) |
| X1 | 1 | 0(0.4) | 0.7 | 0 | 1(0.8) | 0.6 | 1 |
| X2 | 1 | 1(0.8) | 0.7 | 1 | 1(0.7) | 0.6 | 1 |
| X3 | 1 | 0(0.2) | 0.7 | 0 | 1(0.7) | 0.6 | 1 |
| X4 | 1 | 1(0.6) | 0.7 | 0 | 0(0.6) | 0.6 | 1 |

(*1): The numbers in parentheses indicate the estimated probability.
(*2): When estimated probability ≥ threshold value, the value = 1; otherwise the value = 0.

Determining Presence or Absence of Gene Mutation in Undetected Tumor in Patient with Tumor

When, though the presence of gene mutation was determined based on ctDNA (true value), all of the results for the presence or absence of gene mutation output by each corrected radiogenomics model were absence, considering the possibility of detecting ctDNA originated from an undetected tumor, screening for the presence or absence of gene mutation was performed using the radiogenomics model to determine the presence or absence of gene mutation in the undetected tumor.

Determining Therapeutic Effect of Drug on Patient with Tumor

Based on the obtained ctDNA test results and the results of the presence or absence of gene mutation output by each radiogenomics model, the therapeutic effect of the drug on a patient with tumor was determined as effective by confirming no tumor growth after treatment.

From the above, the embodiments disclosed in this description and the drawings are shown to enable analysis of tumor site-specific gene mutation in a subject with multiple tumors, based on gene mutation information identified by ctDNA testing.

At least one of the embodiments described above allows analysis of tumor site-specific gene mutation in a subject with multiple tumors, based on gene mutation information identified by ctDNA testing.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. A method of analyzing gene mutation, comprising:
acquiring first gene mutation information identified by a ctDNA test in a subject, second gene mutation information output by analyzing an image of a first imaging target site in the subject, and third gene mutation information output by analyzing an image of a second imaging target site different from the first imaging target site in the subject; and
determining whether a tumor-specific gene mutation included in the first gene mutation information is associated with a gene mutation in the first imaging target site included in the second gene mutation information, or a gene mutation in the second imaging target site included in the third gene mutation information.

2. The method according to claim 1, further comprising:
correcting information on the presence or absence of gene mutation in an imaging target site included in at least one of the second gene mutation information and the third gene mutation information.

3. The method according to claim 1, wherein the determining comprising:
showing the tumor-specific gene mutation to be present in a new site different from the first imaging target site and the second imaging target site when neither the gene mutation in the first imaging target site nor the gene mutation in the second imaging target site is determined to be associated with the tumor-specific gene mutation.

4. The method according to claim 1, further comprising determining the following: at a first determination time, the tumor-specific gene mutation is determined to be present in any imaging target site; at a second determination time of an arbitrary time after the first determination time, the tumor-specific gene mutation is determined to be not present in the imaging target site; and thus a drug applied to the subject until the second determination time is determined to have a therapeutic effect against a disease associated with the tumor-specific gene mutation.

5. The method according to claim 1 used in a method for treating a tumor in a subject, further comprising:
if determining a tumor-specific gene mutation included in the first gene mutation information is associated with a gene mutation in the first imaging target site included in the second gene mutation information, or a gene mutation in the second imaging target site included in the third gene mutation information, then administering a therapeutically effective amount of drug to the subject determined to have one or more tumors whose presence is (are) confirmed by the gene mutation at the first imaging target site or at the second imaging target site based on the determination data.

6. The method according to claim 5, wherein the subject is a human.

7. The method according to claim 5, wherein the tumor is primary or metastatic.

8. The method according to claim 5, wherein the drug is one or more selected from the group consisting of cytotoxic anticancer drugs, molecular targeted drugs, and immune checkpoint inhibitors.

9. An apparatus for analyzing gene mutation, comprising:
processing circuitry configured to
acquire first gene mutation information identified by a ctDNA test in a subject, second gene mutation information output by analyzing an image of a first imaging target site in the subject, and third gene mutation information output by analyzing an image of a second imaging target site different from the first imaging target site in the subject; and
determine whether a tumor-specific gene mutation included in the first gene mutation information is associated with a gene mutation in the first imaging target site included in the second gene mutation information, or a gene mutation in the second imaging target site included in the third gene mutation information.

10. The apparatus according to claim 9, wherein the processing circuitry is further configured to
control an output configured to output a determination result as to whether the tumor-specific gene mutation included in the first gene mutation information is associated with a gene mutation in the first imaging target site included in the second gene mutation information, or a gene mutation in the second imaging target site included in the third gene mutation information.

11. The apparatus according to claim 10, wherein the processing circuitry is further configured to
control the output configured to show the tumor-specific gene mutation to be present in a new site different from the first imaging target site and the second imaging target site when neither the gene mutation in the first imaging target site nor the gene mutation in the second imaging target site is determined to be associated with the tumor-specific gene mutation.

12. The apparatus according to claim 9, wherein the processing circuitry is further configured to
correct information on the presence or absence of gene mutation in an imaging target site included in at least one of the second gene mutation information and the third gene mutation information.

13. The apparatus according to claim 9, wherein the processing circuitry is further configured to
determine the following: at a first determination time, the tumor-specific gene mutation is determined to be present in any imaging target site; at a second determination time of an arbitrary time after the first determination time, the tumor-specific gene mutation is determined to be not present in the imaging target site; and thus a drug applied to the subject until the second determination time is determined to have a therapeutic effect against a disease associated with the tumor-specific gene mutation.

14. The apparatus according to claim 9, wherein the subject is a human.

15. A non-transitory computer readable storage medium, which stores a program that causes an apparatus for analyzing gene mutation to execute processing comprising:
acquiring first gene mutation information identified by a ctDNA test in a subject, second gene mutation information output by analyzing an image of a first imaging target site in the subject, and third gene mutation information output by analyzing an image of a second imaging target site different from the first imaging target site in the subject; and
determining whether a tumor-specific gene mutation included in the first gene mutation information is associated with a gene mutation in the first imaging target site included in the second gene mutation information, or a gene mutation in the second imaging target site included in the third gene mutation information.

16. The non-transitory computer readable storage medium according to claim 15, which stores the program that causes the apparatus for analyzing gene mutation to execute the processing further comprising:
controlling an output configured to output a determination result as to whether the tumor-specific gene mutation included in the first gene mutation information is associated with a gene mutation in the first imaging target site included in the second gene mutation information, or a gene mutation in the second imaging target site included in the third gene mutation information.

17. The non-transitory computer readable storage medium according to claim 16, wherein the controlling an output shows the tumor-specific gene mutation to be present in a new site different from the first imaging target site and the second imaging target site when neither the gene mutation in the first imaging target site nor the gene mutation in the second imaging target site is determined to be associated with the tumor-specific gene mutation.

18. The non-transitory computer readable storage medium according to claim 15, which stores the program that causes the apparatus for analyzing gene mutation to execute the processing further comprising:
correcting configured to correct information on the presence or absence of gene mutation in an imaging target site included in at least one of the second gene mutation information and the third gene mutation information.

19. The non-transitory computer readable storage medium according to claim 15, wherein the determining further determine the following: at a first determination time, the tumor-specific gene mutation is determined to be present in any imaging target site; at a second determination time of an arbitrary time after the first determination time, the tumor-specific gene mutation is determined to be not present in the imaging target site; and thus a drug applied to the subject until the second determination time is determined to have a therapeutic effect against a disease associated with the tumor-specific gene mutation.

20. The non-transitory computer readable storage medium according to claim 15, wherein the subject is a human.
